(19) United States
(12) Patent Application Publication
(10) Pub. No.: US 2009/0086926 A1
(43) Pub. Date: Apr. 2, 2009

(54) EXPOSURE CENTERING APPARATUS FOR IMAGING SYSTEM

(75) Inventors: Xiaohui Wang, Pittsford, NY (US); David H. Foos, Rochester, NY (US)

Correspondence Address: Susan L. Parulski;Patent Legal Staff, Carestream Health, Inc., 150 Verona Street, Rochester, NY, 14608, US

(73) Assignee: Carestream Health, Inc.

(21) Appl. No.: 11/862,579

(22) Filed: Sep. 27, 2007

(51) Int. Cl. A61B 6/00; G08B 3/00; G08B 5/36

(52) U.S. Cl. 378/206; 340/384.1; 340/815.45; 378/205

(57) ABSTRACT

A radiation imaging system has a radiation source having an adjustable angular orientation and an emitter that provides an alignment signal and is coupled to the radiation source. A two dimensional radiation image detection device has a receiver that records an image according to radiation emitted from the radiation source, a first sensor coupled in a fixed position relative to the receiver that detects the alignment signal from the emitter and provides a first response signal and a second sensor coupled in a fixed position relative to the receiver that detects the alignment signal from the emitter and provides a second response signal. A control logic processor is in communication with the first and second sensors for receiving the first and second response signals and further in communication with at least one indicator for indicating the alignment of the image detection device relative to the radiation source.

### FIELD OF THE INVENTION

This invention relates to an apparatus for radiation imaging, having a positioning apparatus for providing proper alignment of the radiation source relative to an image detection device for recording a radiation image.

### BACKGROUND OF THE INVENTION

When an x-ray image is obtained, there is generally an optimal angle between the radiation source and the two dimensional receiver that records the image data. In most cases, it is preferred that the x-ray source provide radiation in a direction that is perpendicular to the surface of the recording medium. For this reason, large-scale radiography systems mount the radiation head and the recording medium holder at a specific angle relative to each other. Orienting the head and the receiver typically requires a mounting arm of substantial size, extending beyond the full distance between these two components. With such large-scale systems, unwanted tilt or skew of the receiver is thus prevented by the hardware of the imaging system itself.

With the advent of portable radiation imaging apparatus, such as those used in Intensive Care Unit (ICU) environments, a fixed angular relationship between the radiation source and two-dimensional radiation receiver is no longer imposed by the mounting hardware of the system itself. Instead, an operator is required to aim the radiation source toward the receiver surface, providing as perpendicular an orientation as possible, typically using a visual assessment. In computed radiography (CR) systems, the two-dimensional image-sensing device itself is a portable cassette that stores the readable imaging medium.

There have been a number of approaches to the problem of providing methods and tools to assist operator adjustment of source and receiver angle. One classic approach has been to provide mechanical alignment in a more compact fashion, such as that described in U.S. Pat. No. 4,752,948 entitled “Mobile Radiography Alignment Device” to MacMahon. A platform is provided with a pivotable standard for maintaining alignment between an imaging cassette and radiation source. However, complex mechanical solutions of this type tend to reduce the overall flexibility and portability of these x-ray systems. Another type of approach, such as that proposed in U.S. Pat. No. 6,422,750 entitled “Digital X-ray Imager Alignment Method” to Kwasnick et al. uses an initial low-exposure pulse for detecting the alignment grid; however, this method would not be suitable for portable imaging conditions where the receiver must be aligned after it is fitted behind the patient.

Other approaches project a light beam from the radiation source to the receiver in order to achieve alignment between the two. Examples of this approach include U.S. Pat. No. 5,388,143 entitled “Alignment Method for Radiography and Radiography Apparatus Incorporating Same” and No. 5,241,578 entitled “Optical Grid Alignment System for Portable Radiography and Portable Radiography Apparatus Incorporating Same”, both to MacMahon. Similarly, U.S. Pat. No. 6,154,522 entitled “Method, System and Apparatus for Aiming a Device Emitting Radiant Beam” to Cumings describes the use of a reflected laser beam for alignment of the radiation target. However, the solutions that have been presented using light to align the CR cassette or DR receiver are constrained by a number of factors. The '143 and '578 MacMahon disclosures require that a fixed Source-to-Image Distance (SID) be determined beforehand, then use triangulation with this fixed SID value. Changing the SID requires a number of adjustments to the triangulation settings. This arrangement is less than desirable for portable imaging systems that allow a variable SID. Devices using lasers, such as that described in the '522 Cumings disclosure, inherently present some occupational hazard concerns and, in some cases, can require much more precision in making adjustments than is necessary.

Another solution for maintaining a substantially perpendicular relationship between the radiation source and the two-dimensional image detection device is described in U.S. Pat. No. 7,156,553 entitled “Portable Radiation Imaging System and a Radiation Image Detection Device Equipped with an Angular Signal Output Means” to Tanaka et al. In the Tanaka et al. '553 disclosure, an angular sensing device is provided atop or along an edge of the image detection device. The angular sensing device sends a signal to adjust either the tilt angle of the image detection device or the orientation angle of the radiation source in order to maintain a perpendicular relationship of the image detection device to the radiation source. This same approach had previously been used in a number of X-ray products, such as the Siemens Mobilett XP hybrid portable X-ray source, for example, that used built-in tilt sensors.

Similar, then, to these earlier approaches that also used tilt relative to gravity, the solution proposed in the Tanaka et al. '553 disclosure has limited value for achieving alignment between the image sensing device and the radiation source. Measuring tilt with respect to gravity is suitable for one particular case: that is, where the image sensing device is intended to be level and where the radiation source is to be perpendicular to the surface of the image sensing device. In any other orientation, however, solutions of this type become increasingly less effective as the surface of the image sensing device moves away from a perfectly level orientation. There is not enough positioning information with this type of solution for aligning the central ray of the radiation source with the normal to the image sensing device surface. In the worst-case position, with the image-sensing device in a near-vertical or vertical orientation, there is little or no information that can be obtained from tilt sensors as to whether or not the surface of the image sensing device is perpendicular to the radiation source.

Today's portable radiation imaging devices allow considerable flexibility for placement of the CR cassette or Digital Radiography (DR) receiver by the radiology technician. The patient need not be in a horizontal position for imaging, but may be at any angle, depending on the type of image that is needed and the ability to move the patient for the x-ray examination. The technician can manually adjust the position of both the cassette and the radiation source independently for each imaging session. Thus, it can be appreciated that an alignment apparatus for obtaining the desired angle between the radiation source and the surface of the image sensing device must be able to adapt to whatever orientation is best suited for obtaining the image. Tilt sensing, as has been conventionally applied and as is used in the device of the Tanaka et al. '553 disclosure and elsewhere, does not provide sufficient information on cassette-to-radiation source orientation, except in the single case where the cassette is level. More complex position sensing devices can be used, but can be subject to sampling and accumulated rounding errors that can grow worse over time, requiring frequent resynchronization.

Thus, it is apparent that conventional alignment solutions may be workable for specific types of systems and environments; however, considerable room for improvement remains. Portable radiography apparatus must be compact and lightweight, which makes the mechanical alignment approach such as that given in the '948 MacMahon disclosure less than desirable. The constraint to direct line of sight alignment reduces the applicability of many types of reflected light based methods to a limited range of imaging situations. The complex sensor and motion control interaction required by the Tanaka et al. '553 solution would add considerable expense, complexity, weight, and size to existing designs, with limited benefits. Many less expensive portable radiation imaging units do not have the control logic and motion coordination components that are needed in order to achieve the necessary adjustment. None of these approaches gives the operator the needed information for making a manual adjustment that is in the right direction for correcting misalignment.

Significantly, none of these conventional solutions described earlier is particularly suitable for retrofit to existing portable radiography systems. That is, implementing any of these earlier solutions would be prohibitive in practice for all but newly manufactured equipment and could have significant cost impact.

Yet another problem not addressed by many of the above solutions relates to the actual working practices of radiologists and radiological technicians. A requirement for perpendicular delivery of radiation, given particular emphasis in the Tanaka et al. '553 application, is not optimal for all types of imaging. In fact, there are some types of diagnostic images for which an oblique (non-perpendicular) incident radiation angle is most desirable. For example, for the standard chest anterior-posterior (AP) view, the recommended central ray angle is oblique from the perpendicular (normal) by approximately 3-5 degrees. Conventional alignment systems, while they provide for normal incidence of the central ray, do not adapt to assist the technician for adjusting to an oblique angle.

Thus, it can be seen that there is a need for an apparatus that enables proper angular alignment of a radiation source relative to an image detection device for recording a radiation image.

### SUMMARY OF THE INVENTION

An object of the present invention is to provide an alignment apparatus that is particularly suitable for a portable radiation imaging system. Accordingly, the present application discloses a radiation imaging system comprising a radiation source having an adjustable angular orientation; b) an emitter that provides an alignment signal and is coupled to the radiation source; a radiation image detection device having a receiver that records an image according to radiation emitted from the radiation source; a first sensor coupled in a fixed position relative to the receiver that detects the alignment signal from the emitter and provides a first response signal; a second sensor coupled in a fixed position relative to the receiver that detects the alignment signal from the emitter and provides a second response signal; and a control logic processor in communication with the first and second sensors for receiving the first and second response signals and further in communication with at least one indicator for indicating the alignment of the image detection device relative to the radiation source. In another embodiment, the positions of the emitter and sensors are reversed relative to the radiation source and the receiver.

In one embodiment, the radiation imaging system uses timing for receiving a signal transmitted from near the radiation source.

An advantage of the system is that it allows straightforward retrofitting for existing x-ray apparatus.

Another advantage of the system is that it provides a method that can be used with a variable SID distance and can even be used to provide SID measurement in some embodiments.

These and other objects and advantages of the present invention will become apparent to those skilled in the art upon reading the following detailed description when taken in conjunction with the drawings wherein there is shown and described an illustrative embodiment of the invention.

### BRIEF DESCRIPTION OF THE DRAWINGS

While the specification concludes with claims particularly pointing out and distinctly claiming the subject matter of the present invention, it is believed that the invention will be better understood from the following description when taken in conjunction with the accompanying drawings, wherein:

FIG. 1 is a perspective view showing angles and coordinates of interest for alignment of a radiation source to a receiver, showing grid orientation;

FIG. 2 is a plan view showing the sensor housing of the present invention from the radiation source;

FIG. 3 is a plan view showing control and display components for the alignment apparatus of the present invention;

FIG. 4 is a perspective view showing alignment components;

FIG. 5A is a timing diagram for a synchronized embodiment;

FIG. 5B is a timing diagram for an asynchronous embodiment;

FIGS. 6A and 6B are side and front views of the alignment component housing of the present invention in one embodiment;

FIG. 7 is a perspective view of an alternate embodiment using wireless communication; and

FIGS. 8A and 8B show perspective views of operation of another embodiment that uses two transmitters and a single sensor.

### DETAILED DESCRIPTION OF THE INVENTION

Unlike the limited tilt sensing approaches that have been used in a variety of earlier radiography systems, the apparatus and method of the present invention provide a straightforward solution to the problem of radiation source-to-receiver alignment that can be used with a number of CR and DR imaging systems. The present invention uses a form of triangulation to determine proper source angle.

Figures and timing diagrams in the present disclosure are provided in order to show concepts and important components more clearly and are not drawn with attention to scale.

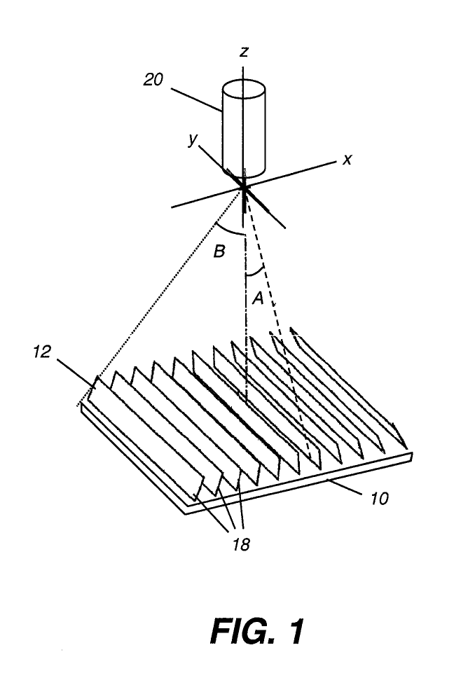

FIG. 1 shows angles and coordinates of interest for alignment of source to receiver. A radiation source 20 having an adjustable angular orientation is shown in position for directing radiation toward a receiver 10, such as a film cassette, CR cassette or DR receiver. Receiver 10 may have a photostimulable medium, such as a film or phosphor medium, for example, or may have a detector array that records an image according to radiation emitted from radiation source 20. An antiscatter grid 12 has plates 18 arranged as shown in FIG. 1, just above the surface of the receiver 10. Coordinate xyz axes are shown, with the source-to-image distance (SID) in the direction of the z axis. Angle A is in the yz plane, parallel to the length direction of grid 12 plates. Angle B is in the xz plane, orthogonal to the length direction of grid 12 plates. Angle A can vary over some range, since it is in parallel with grid 12 plates. Angle B, however, is constrained to a narrower range, typically within about +/−5 degrees of normal.

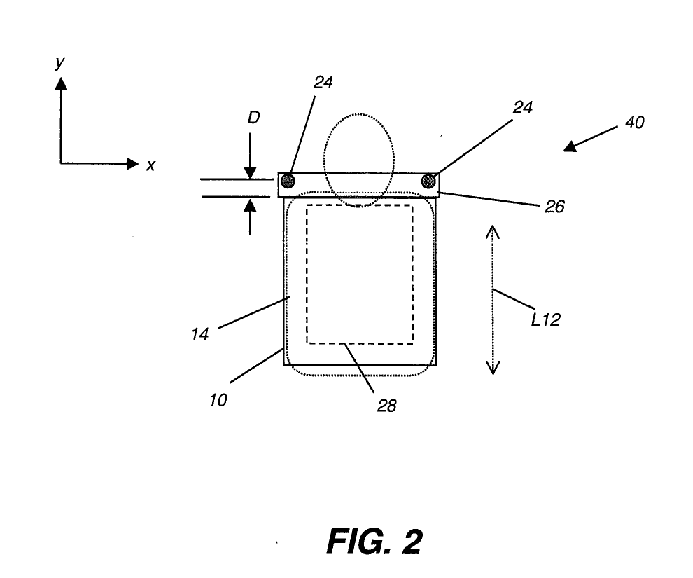

FIG. 2 shows how an alignment apparatus 40 according to the present invention can be positioned for use. Receiver 10 is placed in position behind a patient 14. Line L12 shows the grid direction. A housing 26 has been fitted onto receiver 10 and holds two sensors 24 and, optionally, other components of alignment apparatus 40. Sensors 24 are suitably coupled in fixed, symmetric positions in relation to housing 26.

In practice, receiver 10 is placed behind the patient with the patient anatomy to be imaged centered relative to the receiver. Sensors 24 are mounted on top of the receiver facing source 20 and disposed symmetrically. Because receiver 10 is placed behind the patient for imaging, it may not be visible to the x-ray operator when in position for imaging. However, sensors 24 are mounted outboard of receiver 10, such as high enough such that a clear line of sight is available between sensors 24 and radiation source 20. Using a chest exam as an example, sensors 24 are visible near the neck of the patient.

By design, sensors 24 are a known distance D above the top of receiver 10 as shown for example in FIG. 2. Because of this, the x-ray operator can use sensors 24 as reference targets, knowing distance D, for estimating the location of receiver 10 behind the patient. The operator can then properly aim, position, and center the collimation field (as indicated by a collimator light pattern 28) on the patient. This provides the initial setup that is needed for centering radiation source 20, the x-ray tube, for imaging. Once this setup is performed, a method described subsequently is used to facilitate centering, using the triangulation method of the present invention. When both the collimator light and the x-ray tube of radiation source 20 are centered relative to sensors 24, suitable alignment between receiver 10 and the incident x-ray beam is achieved.

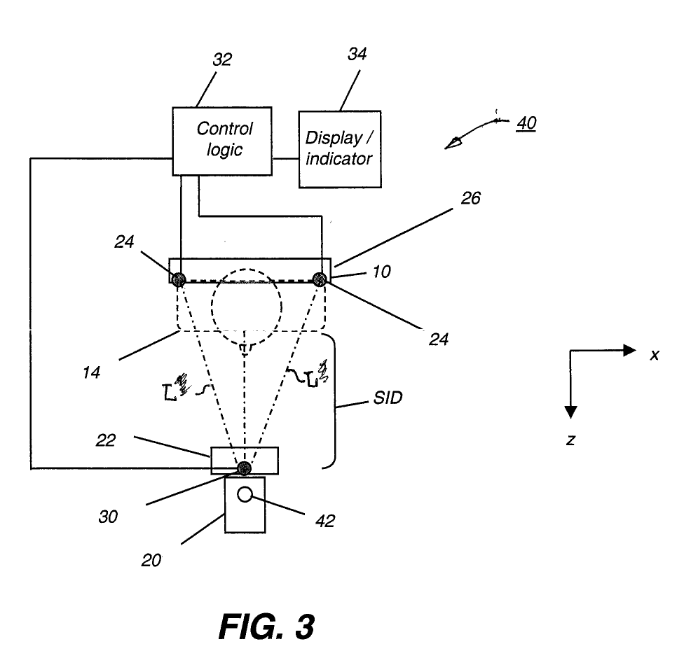

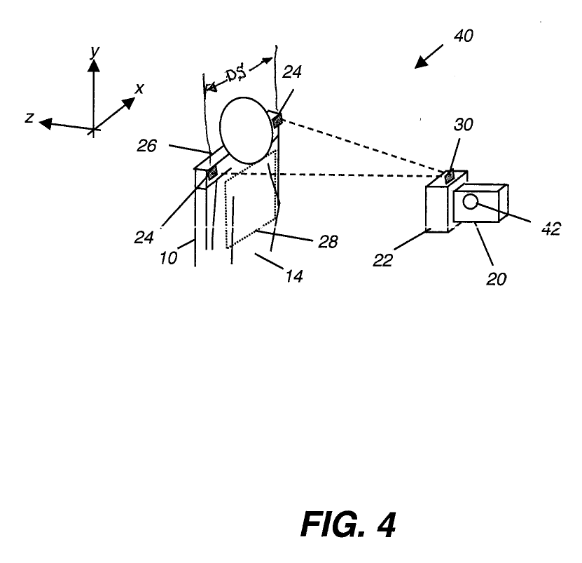

The views of FIG. 3 and of FIG. 4 show how alignment apparatus 40 of the present invention applies triangulation principles to the problem of aligning receiver 10 to radiation source 20. An emitter 30, mounted on or near a collimator 22 in the embodiment shown, emits a signal that is detected by sensors 24. A control logic processor 32 monitors the response of sensors 24 to determine alignment conditions. An indicator or display 34 then reports alignment results to aid the operator in making any necessary adjustments. As shown in FIG. 2 and FIG. 4, light pattern 28 from a collimator light 42 associated with collimator 22 can be properly aimed when housing 26 is in place on receiver 10.

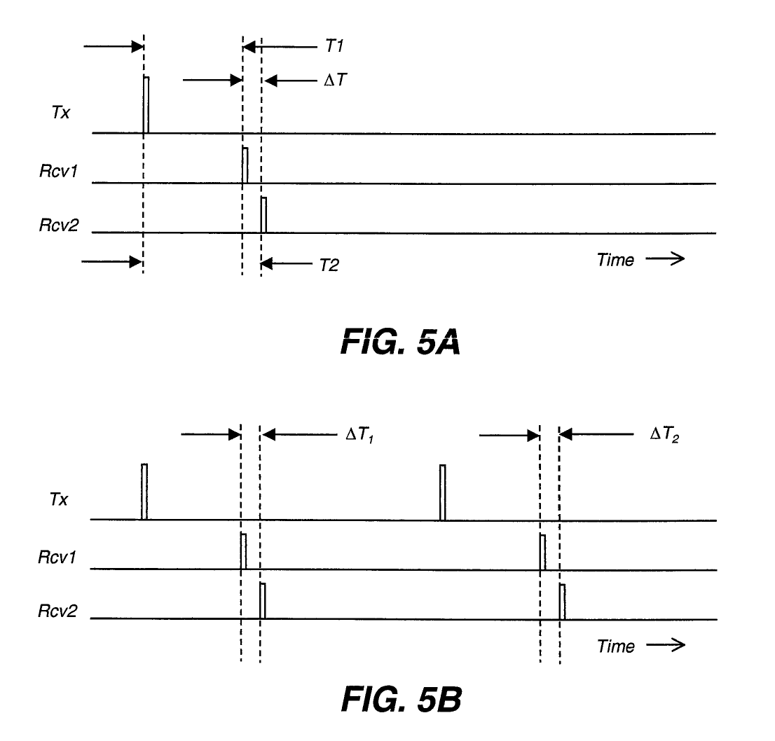

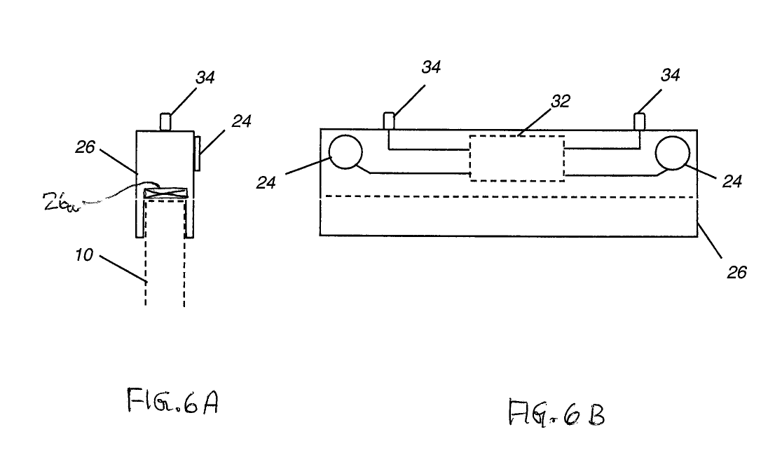

Referring again to FIG. 3, emitter 30 emits the signal detected by sensors 24. In one embodiment, the emitted signal is an ultrasound signal. Emitter 30 may be in communication with control logic processor 32 for synchronous operation, as shown in the embodiment of FIG. 3, or may be separately controlled for asynchronous operation. In synchronous operation, as shown in the timing diagram of FIG. 5A, both the SID and receiver alignment can be readily checked. The Tx timing shows a pulsed signal transmitted from emitter 30. Rcv1 and Rcv2 timing shows the same pulsed signal received at first and second sensors 24 which may be shown to the operator on display 34. Time T1 and T2 can be used by control logic 32 to compute the approximate SID, simply using:

L=s×(T1+T2)/2

SID=sqrt(L²−(DS/2)²)

wherein s is the speed of the signal emitted, such as the speed of sound for an ultrasound signal. DS is the distance between the sensors 24. As computed in accordance with the above relationships, L is the averaged distance between emitter 30 and either sensor 24. Thus, an approximate value of SID is determined by control logic using the familiar Pythagorean theorem and displayed to the operator.

Still referring to FIG. 5A, time ΔT can be used to compute the relative alignment angle offset of receiver 10 with respect to normal. Ideally, time ΔT is zero; in practice, some slight angular error is acceptable, within no more than about +/−4 or +/−5 degrees. As an example of the scale of this measurement, where the SID is approximately one meter and ultrasound is used, time T1 is 1/344 second, about 2.9 microseconds. For an angular error of about 2 degrees using this SID with a standard sized CR cassette, time T2 is on the order of about 0.035 microseconds.

FIG. 5B shows alternate timing relationships when control logic processor 32 is not in communication with emitter 30 and asynchronous operation is used. Here, the Tx pulse timing is not itself sensed. Instead, only the difference time ΔT between received pulses Rcv1 and Rcv2 is used. Two example intervals, ΔT₁ and ΔT₂ are shown. Since precision angular alignment is not required and some reasonable tolerance is allowable, reducing time ΔT below a predetermined threshold value may be all that is needed for an asynchronous application.

For either synchronous or asynchronous operation, emitter 30 can be triggered by activation of the collimator light that provides collimator light pattern 28. In asynchronous operation, simply turning collimator light 42 on would then cause emitter 30 to periodically emit a pulse for aiding alignment as shown in FIG. 5B. In synchronous operation, turning on collimator light 42 could cause emitter 30 to be synchronized and controlled by control logic processor 32.

As noted in the timing examples of FIGS. 5A and 5B, ultrasound can be used as the emitted signal for this application. Ultrasound has a number of advantages over other signal types and can be readily detected by relatively inexpensive sensors. There is no interference between the ultrasound signal and any emitted radiation and only very low energy levels are needed. Pulsed timing, as shown in the timing examples of FIGS. 5A and 5B, is particularly appropriate since transition detection is all that is necessary. Because of the tolerance for error, highly precise sensor resolution is not needed and the support electronics can be fairly inexpensive. In one embodiment, the ultrasound transmitter used as emitter 30 is a model SRF05 available from Acroname, Inc., Boulder, Colo. Receivers 24 are also model SRF05. Other types of wireless signal, such as RF signals, infrared signals, electronic signals, and the like could alternately be used.

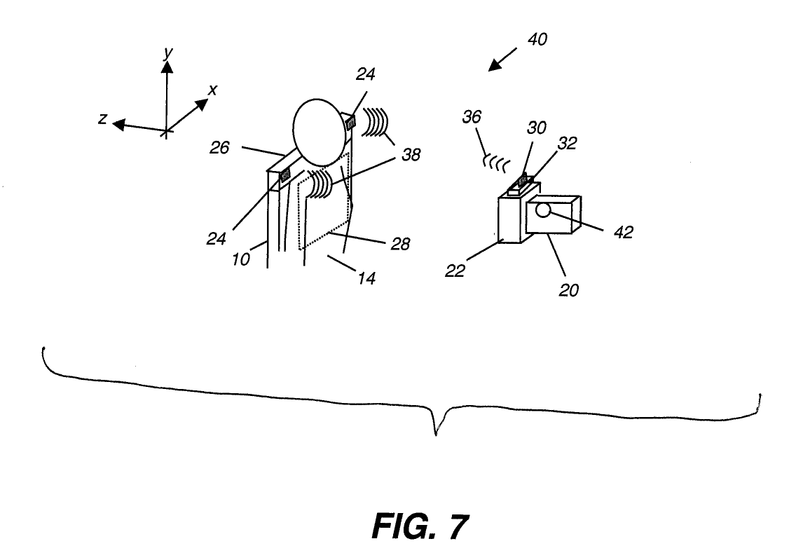

FIGS. 6A and 6B show side and front views of housing 26 in one embodiment. Here, control logic processor 32 is packaged within housing 26 along with other support components, not shown, such as battery or power supply, for example. Sensors 24 mount on the front of housing 26 and indicator 34 components are LEDs or lamps that illuminate to indicate the relative status of alignment. For example, both lights may be illuminated when a normal alignment has been achieved by the operator. As shown in FIG. 6A, housing 26 is fitted onto the CR cassette or other receiver 10. Any number of fitting arrangements could be used, as is well known in the mechanical arts, in order to suitably mount housing 26 to the CR cassette or other receiver 10 during alignment and imaging, such as a magnetic coupling 26a.

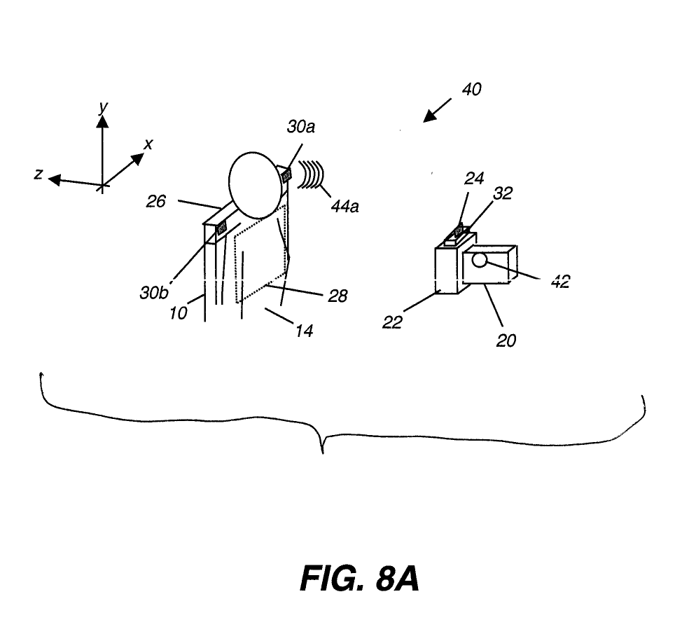

A number of other packaging arrangements are possible for alignment apparatus 40. For example, sensors 24 could be separately clipped onto opposite edges of receiver 10 and wirelessly connected to control logic processor 32. Referring to FIG. 7, control logic processor 32 could be provided as part of a package that includes emitter 30 and mounts on or near collimator 22. Wired or wireless communication could then be used between sensors 24 and control logic processor 32 mounted on collimator 22. For example, Bluetooth or other RF communication could be used between sensors 24 and control logic processor 32. The embodiment shown in FIG. 7 sends a first signal 36, ultrasound, as its sensed signal for alignment and distance. In response, sensors 24 return an RF signal 38 as the communication signal for obtaining the needed timing information.

Where ultrasound is used as the signal for checking alignment, it can be useful to check that either or both sensors 24 are not blocked. This can be accomplished by sensing the amplitude of the received signal. A signal that is below a predetermined amplitude threshold will indicate that the path of the emitted signal is blocked. In such a case, an error condition can be displayed or otherwise made known to the operator.

Indicator 34 can take any of a number of forms. In the embodiment of FIG. 6, for example, an LED or other indicator could illuminate to indicate whether or not alignment is acceptable. A multi-color indicator could be used, for example, emitting one color for an error condition, another color when alignment is within an acceptable range. Multiple LEDs, lamps, or other indicators could be used, displaying various patterns that indicate whether or not alignment is acceptable and may also indicate in which direction adjustment is needed.

In yet other embodiments, a display monitor can be used as indicator 34. For example, a display monitor that acts as the interface to the X-ray system may be used to display the additional alignment information, as well as SID information that can be obtained from the responding sensors. A symbol, such as an icon or alphanumeric text or message, can be provided to indicate alignment status. An audible indicator could alternately be used. For example, an indicator could emit a beep or other tone to indicate alignment status.

In another embodiment, the SID information is transferred to the image capture device when the images are scanned and is combined with other patient information as part of Digital Imaging and Communications in Medicine (DICOM) output. Further, if the SID information can be obtained from the capture device for the previously acquired images, this SID can be used as the aim for subsequent imaging. Referring back to FIG. 3, control logic processor 32 could be programmed to use indicator 34 to inform the x-ray operator of needed SID adjustment.

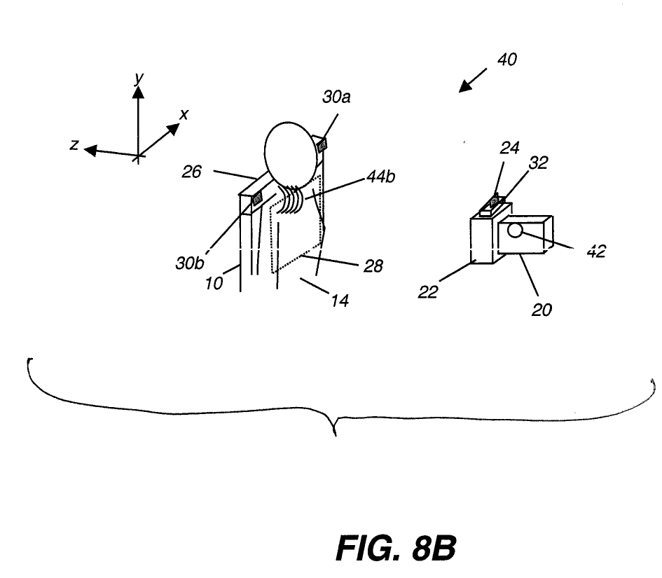

FIGS. 8A and 8B show an alternate embodiment in which the positions of sensor and emitter components are reversed. Here, housing 26 is coupled to two emitters 30a and 30b and sensor 24 is coupled to collimator 22. Emitter 30a sends a signal 44a that alternates with a signal 44b that is transmitted from emitter 30b. The relative timing of the two emitted signals 44a and 44b is then used to measure alignment between radiation source 20 and receiver 10. Alternately, the two signals 44a and 44b could be sent at the same time, or in some specified sequence, so that a difference between signal timing can provide alignment offset information.

### Example Operation Sequence

The basic sequence for obtaining a suitable alignment between receiver 10 and radiation source 20 (FIGS. 1, 3, 4) using alignment apparatus 40 of the present invention is as follows:
1. Position sensors 24 on receiver 10. This may simply require attaching housing 26 (FIG. 6) or other assembly that holds sensors 24 onto receiver 10. Then, position receiver 10 behind the patient and center the receiver to the anatomical region to be examined.
2. Aim collimator light 42 onto the area of the patient that is to be imaged, using sensor 24 or other markings provided as part of alignment apparatus 40 as a visual guide. Adjust collimator settings accordingly, using collimator light pattern 28 as a guide. This step ensures that the radiation field is centered with receiver 10 in position behind the patient.
3. Observe indicator 34 to determine whether the desired radiation head normal alignment has been obtained. Adjust the x-ray tube or other radiation head of radiation source 20 accordingly until indicator 34 indicates suitable positioning while maintaining collimator light pattern 28 position on the patient.
4. Obtain the image.

Unlike the limited alignment approaches that have been used in a variety of earlier radiography systems, the apparatus and method of the present invention work with a variable SID, not requiring readjustment of targets or other manipulation when changing this distance. The alignment apparatus of the present invention can be retrofit to existing digital radiography systems, both CR and DR, as well as to earlier film-based radiography apparatus. The alignment apparatus can be readily adjusted for calibration and does not require periodic reset.

The invention has been described in detail with particular reference to certain preferred embodiments thereof, but it will be understood that variations and modifications can be effected within the scope of the invention as described above, and as noted in the appended claims, by a person of ordinary skill in the art without departing from the scope of the invention. For example, any of a number of different methods could be used for mechanically coupling emitter 30 to collimator 22. A press-fit or magnetic coupling could be used. Indicator 34 can be as simple as a single LED or lamp or can use a set of multiple LEDs or a portion of a display monitor screen.

Thus, what is provided is an apparatus and method for providing proper alignment of the radiation source relative to an image detection device for recording a radiation image.

### PARTS LIST

10. Receiver
12. Grid
14. Patient
18. Plates
20. Radiation source
22. Collimator
24. Sensor
26. Housing
26a. Magnetic coupling
28. Collimator light pattern
30, 30a, 30b. Emitter
32. Control logic processor
34. Indicator
36. Signal
38. Signal
40. Alignment apparatus
42. Collimator light
44a, 44b. Signal
A, B. Angle
D. Distance
L12. Line of grid direction
DS. Distance between sensors 24

1. A radiation imaging system comprising:
a radiation source having an adjustable angular orientation;
an emitter that provides an alignment signal and is coupled to the radiation source;
a radiation image detection device comprising a receiver that records an image according to radiation emitted from the radiation source;
a first sensor coupled in a fixed position relative to the receiver that detects the alignment signal from the emitter and provides a first response signal;
a second sensor coupled in a fixed position relative to the receiver that detects the alignment signal from the emitter and provides a second response signal; and
a control logic processor in communication with the first and second sensors for receiving the first and second response signals and further in communication with at least one indicator for indicating the alignment of the image detection device relative to the radiation source.

2. The radiation imaging system of claim 1 wherein the sensor apparatus is encased in a housing that is detachable from the cassette.

3. The radiation imaging system of claim 1 wherein the at least one indicator provides an audible signal.

4. The radiation imaging system of claim 2 wherein the housing comprises a magnetic coupling.

5. The radiation imaging system of claim 1 wherein the emitter provides an ultrasound signal.

6. The radiation imaging system of claim 1 wherein the emitter provides an RF signal.

7. The radiation imaging system of claim 1 wherein the first response signal is an RF signal.

8. The radiation imaging system of claim 1 wherein the emitter provides an infrared signal.

9. The radiation imaging system of claim 1 wherein the first response signal is an infrared signal.

10. The radiation imaging system of claim 1 wherein the at least one indicator displays a numerical value.

11. The radiation imaging system of claim 1 wherein the at least one indicator comprises a display monitor.

12. The radiation imaging system of claim 1 wherein the at least one indicator is an LED.

13. The radiation imaging system of claim 1 wherein the at least one indicator is a lamp.

14. The radiation imaging system of claim 1 wherein the first response signal is an electronic signal.

15. The radiation imaging system of claim 1 wherein the at least one indicator is a plurality of LEDs that indicate relative alignment.

16. An alignment apparatus for a radiation imaging system having a radiation source and an image detection device, the apparatus comprising:
an emitter that provides an alignment signal and is adapted to be coupled to the radiation source of the imaging system;
a first sensor adapted to be coupled to the detection device that detects the alignment signal from the emitter and provides a first response signal;
a second sensor adapted to be coupled to the detection device that detects the alignment signal from the emitter and provides a second response signal; and
a control logic processor in communication with the first and second sensors for receiving the first and second response signals and further in communication with at least one indicator for indicating the alignment of the image detection device relative to the radiation source.

17. A method for obtaining alignment to a target in a radiation imaging system comprising:
a) aiming a radiation source toward the target;
b) emitting an alignment signal from an emitter coupled to the radiation source;
c) detecting the alignment signal from the emitter and providing a first response signal from a sensor coupled toward a first side of a receiver;
d) detecting the alignment signal from the emitter and providing a second response signal from a sensor coupled toward a second side of the receiver;
e) computing an alignment offset according to the timing difference between detection of the first and second response signals;
f) indicating the status of the alignment according to the alignment offset.

18. A radiation imaging system comprising:
a) a radiation source having an adjustable angular orientation;
b) a sensor that detects first and second alignment signals and is coupled to the radiation source;
c) a first emitter coupled in a fixed position relative to a radiation receiver to emit the first alignment signal;
d) a second emitter coupled in a fixed position relative to the radiation receiver to emit the second alignment signal;
e) a control logic processor in communication with the sensor in order to compute alignment based on the relative timing of detection of the first and second alignment signals; and,
f) at least one indicator in communication with the control logic processor for indicating the computed alignment.

19. A method for obtaining alignment to a receiver in a radiation imaging system comprising:
a) aiming a radiation source toward the receiver;
b) emitting a first alignment signal from a first emitter coupled to the receiver;
c) emitting a second alignment signal from a second emitter coupled to the receiver;
d) sensing the first and second alignment signals at a sensor that is coupled to the radiation source;
e) computing an alignment offset according to the timing difference between sensing of the first and second alignment signals;
f) indicating the status of the alignment according to the alignment offset.
